SEQUENCE LISTING (18 sequences)

SEQ ID NO: 1 - DNA, 20 bp, Homo sapiens
ggaagagggaaaagcttcac

SEQ ID NO: 2 - DNA, 19 bp, Homo sapiens
gagtagttgggggaggtgg

SEQ ID NO: 3 - DNA, 25 bp, Homo sapiens
ctggcatctacggggatacgcatca

SEQ ID NO: 4 - DNA, 25 bp, Danio rerio
acttttctccataccttgttgttga

SEQ ID NO: 5 - DNA, 20 bp, Danio rerio
gtcatcgccagctttctacc

SEQ ID NO: 6 - DNA, 21 bp, Danio rerio
ctttgcgattactgaccaacg

SEQ ID NO: 7 - DNA, 20 bp, Danio rerio
gtcgtcctatagacacagtc

SEQ ID NO: 8 - DNA, 21 bp, Danio rerio
ttctggtagatggacgtggag

SEQ ID NO: 9 - DNA, 20 bp, Danio rerio
ctttctgaaggccatcgagc

SEQ ID NO: 10 - DNA, 21 bp, Danio rerio
cagagtccgtcctgaccaaac

SEQ ID NO: 11 - DNA, 21 bp, Danio rerio
ggaacagtatgggatgtatcc

SEQ ID NO: 12 - DNA, 20 bp, Danio rerio
gcaggatctcgttcttgctg

SEQ ID NO: 13 - DNA, 21 bp, Danio rerio
gagatctatcgaccaccaatg

SEQ ID NO: 14 - DNA, 20 bp, Danio rerio
ctggaaagcgatgcacactg

SEQ ID NO: 15 - DNA, 21 bp, Danio rerio
gctaaggcagatccatccctg

SEQ ID NO: 16 - DNA, 21 bp, Danio rerio
cacttctgtagcaggcgatcc

SEQ ID NO: 17 - DNA, 20 bp, Danio rerio
aggcttctcacaaacgagga

SEQ ID NO: 18 - DNA, 20 bp, Danio rerio
gatggccacaatctccactt

(19) United States
(12) Patent Application Publication
(10) Pub. No.: US 2007/0186288 A1
(43) Pub. Date: Aug. 9, 2007

(54) Zebrafish models of acute myelogenous leukemia

(76) Inventors: Randall T. Peterson, Belmont, MA (US); Jing-Ruey Joanna Yeh, Winchester, MA (US)

Correspondence Address: CLARK & ELBING LLP, 101 FEDERAL STREET, BOSTON, MA, 02110, US

(21) Appl. No.: 11/493,959

(22) Filed: Jul. 27, 2006

Related U.S. Application Data
(60) Provisional application No. 60/702,806, filed on Jul. 27, 2005.

(51) Int. Cl. A01K 67/027

(52) U.S. Cl. 800/3; 800/20

(57) ABSTRACT

The invention provides zebrafish models of acute myelogenous leukemia (AML), as well as methods of using these models to identify therapeutic agents for treating AML.

### CROSS-REFERENCE TO RELATED APPLICATIONS

This application claims priority from U.S. Ser. No. 60/702,806, filed Jul. 27, 2005, the contents of which are incorporated herein by reference.

### BACKGROUND OF THE INVENTION

This invention relates to zebrafish models of acute myelogenous leukemia. Acute Myelogenous Leukemia (AML)

AML is the most common form of leukemia. In the United States, more than ten thousand new cases of AML are reported each year. With current chemotherapy regimens, the five year survival rates for AML are only 25-30% for adults younger than 60 and 5-15% for adults older than 60 (Stone et al., Hematology (Am. Soc. Hematol. Educ. Program):98-117, 2004). AML is often associated with chromosomal translocations that generate transcription factor fusion proteins with aberrant function in hematopoietic programming (Scandura et al., Oncogene 21:3422-3444, 2002). As a result, AML patients manifest accumulation of immature hematopoietic blast cells and reduced production of normal marrow cells.

Up to 30% of de novo AML cases can be linked to chromosomal rearrangements in two genes, AML1 (also known as CBFα2, RUNXI, and PEBPαB) and CBFβ. Normally, AML1 and CBFβ form a complex called the core-binding factor (CBF) complex. This complex binds to the enhancer core motif and activates tissue-specific expression of a number of hematopoietic genes, including those encoding the T cell antigen receptors, many of the primary granule proteins in myeloid cells, and a variety of cytokines and their receptors (Lutterbach et al., Gene 245:223-235, 2000; Borregaard et al., Curr. Opin. Hematol. 8:23-27, 2001). The CBF complex also interacts and synergizes with other transcription factors such as PU.1, MEF, and C/EBPβ (Mao et al., Mol. Cell. Biol. 19:3635-3644, 1999; Petrovick et al., Mol. Cell. Biol. 18:3915-1325, 1998; Zhang et al., Mol. Cell. Biol. 16:1231-1240, 1996 ). Multiple chromosomal rearrangements associated with AML involve the genes encoding the CBF complex, suggesting an important role of this complex in maintaining hematopoietic homeostasis.

A reciprocal chromosomal translocation at t(8;21)(q22;q22) is found in approximately 12-15% of all AML cases. This event results in a fusion between the DNA-binding domain of AML-1 and the full-length ETO (for eight twenty-one; also known as MTG8) protein. Since ETO can recruit the nuclear receptor co-repressor (N-CoR)/mSin3/histone deacetylase (HDAC) complex (Licht, Oncogene 20:5660-5679, 2001), the AML1-ETO fusion protein is thought to repress transcription of the genes that are normally activated by the CBF complex. Moreover, this fusion protein may have additional activities other than antagonizing AML1 function (Okuda et al., Blood 91:3134-3143, 1998; Shimada et al., Blood 96:655-663, 2000). However, the identities of the target genes and their roles in AML pathogenesis remain poorly understood.

Recent studies have shown that AML1-ETO influences the activities or expression of several genes with potential relevance in myeloid leukemogenesis. For example, AML1-ETO directly binds to the myeloid master regulator PU.1 and inhibits its transcriptional activity (Vangala et al., Blood 101:270-277, 2003). AML1-ETO was also shown to up-regulate TIS11b, which induces myeloid cell proliferation when overexpressed, and to downregulate the granulocytic differentiation factor C/EBPβ (Shimada et al., Blood 96:655-663, 2000; Pabst et al., Nat. Med. 7:444-451, 2001). γ-catenin (plakoglobin) expression is induced by AML1-ETO, and transfection of γ-catenin into myeloid cells enhances proliferation and prevents maturation during colony growth (Muller-Tidow et al., Mol. Cell. Biol. 24:2890-2904, 2004). AML1-ETO interacts with HEB (HeLa E-box-binding protein) and blocks HEB-dependent transcriptional activation by converting HEB from a transactivator to a potent transcriptional repressor (Zhang et al., Science 305:1286-1289, 2004). Therefore, PU.1, TIS11b, γ-catenin, HEB, and components of the N-CoR/mSin3/HDAC complex are among the molecules that may mediate the effects of AML1-ETO. However, it is not known if any of these molecules are required for leukemogenesis or if any of them are potential targets that can be used to reverse the disease. It is of great importance to clarify the roles of candidate molecules in AML leukemogenesis and to determine whether they may be potential therapeutic targets for the disease. It is also critical that testing of candidate genes be performed in a relevant physiological context. Thus, an AML1-ETO animal model that is amenable to systematic testing of disease modifiers is needed.

Numerous mouse models have been generated to elucidate the molecular mechanisms by which AML1-ETO promotes leukemogenesis (de Guzman et al., Mol. Cell. Biol. 22:5506-5517, 2002; Yuan et al., Proc. Natl. Acad. Sci. U.S.A. 98:10398-10403, 2001; Higuchi et al., Cancer Cell 1:63-74, 2002; Grisolano et al., Proc. Natl. Acad. Sci. U.S.A. 100:9506-9511, 2003). However, these mouse models may not be ideal for identifying or testing disease modifiers due to their low penetrance, long latency, and the relative difficulty of genetic manipulation in mice. Thus, an experimentally tractable model of AML in which the disease phenotype develops quickly and reproducibly, and in which gene expression can be easily manipulated, would greatly facilitate studies of the pathways governing AML pathogenesis and the testing of potential AML therapies.

Small Molecules as Cancer Chemotherapeutics

The majority of cancer chemotherapies involve the use of nonspecific cytotoxic agents that kill proliferating cells indiscriminately. These compounds can be effective at slowing or reversing disease progression, but they typically cause significant toxicity to healthy, non-transformed cells, which limits their efficacy. For decades, the replacement of nonspecific cytotoxic agents with therapies that specifically target the underlying causes of cancer has been viewed as a central goal in cancer research (Sawyers, Nature 432:294-297, 2004; Van Dyke et al., Cell 108:135-144, 2002). The first therapies to achieve this goal have recently begun to come into use. For example, chronic myeloid leukemia (CML) can be caused by translocations resulting in formation of the BCR-ABL fusion gene. Gleevec (imatinib mesylate) inhibits the BCR-ABL protein tyrosine kinase and is effective for treating CML (O'Brien et al., N. Engl. J. Med. 348:994-1004, 2003). Acute promyelocytic leukemia (APL) is a subtype of AML caused by translocations involving the retinoic acid receptor RARα. All-trans retinoic acid is highly effective at treating acute promyelocytic leukemia and has transformed the disease from one of the most fatal subtypes of AML to one that is curable in 70-80% of those affected (Tallman, Semin. Hematol. 41:27-32, 2004).

Gleevec and retinoic acid clearly illustrate the potential of targeted therapies in cancer chemotherapy. However, despite these successes, targeted therapies do not yet exist for most cancers, including the non-APL forms of AML. Development of such therapies is prevented either because the molecular defects underlying those cancers are poorly understood or because of the difficulty of identifying drug targets that can effectively compensate for those defects. Novel approaches for identifying targeted cancer chemotherapeutics are needed.

Phenotype-Based Screens

Recent advances in synthetic chemistry, robotics, and the development of efficient assays have made it possible to ascertain the biological activity of thousands of chemical compounds simultaneously, in a process known as high-throughput screening (HTS). When a therapeutic target has been identified and validated, HTS based upon target binding or function can often be used to identify novel structures that modify the activity of a target protein (Bleicher et al., Nat. Rev. Drug Discov. 2:369-378, 2003). However, this approach is only effective when a valid therapeutic target has been identified (Lindsay, Nat. Rev. Drug Discov. 2:831-838, 2003). Developing therapies for many of the most significant diseases, including AML, is limited by the fact that effective targets have not yet been identified for these diseases, as noted above. In vitro enzymatic assays are often poor surrogates for complex physiological diseases.

One alternative to in vitro target-based drug discovery is discovery guided by phenotype in the context of a whole organism. Whereas target-based approaches can discover compounds that modify a target but may not modify the disease, phenotype-based approaches discover compounds that modify the disease phenotype, without regard to the specific molecular target (Yeh et al., Dev. Cell 5:11-19, 2003; Stockwell, Nat. Rev. Genet. 1:116-125, 2000). This phenotype-based screening approach is often referred to as ‘chemical genetics’ because it borrows from the logic of genetics in which phenotype-based screening is used to discover novel genes affecting a process of interest. Development of many drugs in use today was guided by phenotype analysis of whole organisms (Zon et al., Nat. Rev. Drug Discov. 4:35-44, 2005). For diseases such as AML, for which validated therapeutic targets have not been identified, phenotype-based screens are a promising approach for the discovery of novel therapies.

Zebrafish Animal Model Systems

The zebrafish has emerged as a powerful tool for phenotype-based screens (Anderson et al., Nat. Genet. 33(Suppl.):285-293, 2003; Grunwald et al., Nat. Rev. Genet. 3:717-724, 2002; Patton et al., Nat. Rev. Genet. 2:956-966, 2001). Its genome and body plan are similar to those of other vertebrates, but its optical transparency and external development make real time observation of its internal organs simple. The optical clarity of the zebrafish embryo becomes even more useful when combined with fluorescent markers that highlight the locations or activities of specific populations of cells. For example, dozens of transgenic zebrafish lines have been created which express fluorescent proteins in locations ranging from the presomitic mesoderm (Gajewski et al., Development 130:4269-4278, 2003) to the pituitary gland (Liu et al., Mol. Endocrinol. 17:959-966, 2003). These lines greatly facilitate detection of anatomical changes caused by small molecules. Numerous zebrafish disease models ranging from congenital heart defects to cancers have been developed (Penberthy et al., Front. Biosci. 7:1439-1453, 2002; Amatruda et al., Cancer Cell. Hum. Genet. 3:311-340, 2002; Shin et al., Annu. Rev. Genomics Hum. Genet. 3:311-340, 2002), and the zebrafish is genetically and pharmacologically similar to humans (Langheinrich, Bioessays 25:904-912, 2003; Milan et al., Circulation 107:1355-1358, 2003).

The ease with which zebrafish phenotypes can be identified has resulted in their use in numerous genetic and chemical screens (Anderson et al., Nat. Genet. 33(Suppl.):285-293, 2003; Macrae et al., Chem. Biol. 10:901-908, 2003). Further, because screening can be performed in the whole organism, perturbation of potential therapeutic targets by small molecules or mutations reveals the effects of such perturbations on the integrated physiology of the entire organism. As zebrafish have become more widely used, additional technologies have been developed that have increased the utility of the system even further. The zebrafish genome project is now nearly complete, and DNA microarrays have been generated for expression profiling studies (Ton et al., Biochem. Biophys. Res. Commun. 296:1134-1142, 2002; Stickney et al., Genome Res. 12:1929-1934, 2002). Antisense morpholino oligonucleotides have proven to be an effective means of “knocking down” gene function (Nasevicius et al., Nature Genetics 26:216-220, 2000). More recently, reverse genetic approaches have been developed for the zebrafish, enabling researchers to generate mutations in virtually any gene of interest (Wienholds et al., Science 297:99-102, 2002). Thus, the zebrafish is rapidly becoming a mature model organism, armed with an impressive collection of genomic and experimental tools. These tools are also broadening the scope of whole-organism chemical screens that can be imagined.

Zebrafish Chemical Genetics

The unique attributes of the zebrafish embryo allow chemical genetic technologies to be applied to complex diseases such as leukemia. Unlike yeast, flies, and worms, which are. generally resistant to small molecule permeation, zebrafish embryos readily absorb small molecules from the surrounding medium. Furthermore, their transparency and small size enable screening on a scale that would be prohibitive for mice or other vertebrate model organisms. Zebrafish high-throughput chemical screens have been used to identify potent, specific small molecule modifiers of many aspects of vertebrate development (MacRae et al., Chem. Biol. 10:901-908, 2003; Moon et al., J. Am. Chem. Soc. 124:11608-11609, 2002; Khersonsky et al., J. Am. Chem. Soc. 125:11804-11805, 2003; Peterson et al., Proc. Natl. Acad. Sci. U.S.A. 97:12965-12969, 2000; Peterson et al., Current Biology 11:1481-1491, 2001; Spring et al., J. Am. Chem. Soc. 124:1354-1363, 2002; Stemson et al., J. Am. Chem. Soc. 123:1740-1747, 2001) and to discover novel compounds that suppress disease phenotypes (Peterson et al., Nat. Biotechnol. 22:595-599, 2004).

Two types of zebrafish small molecule screens have been carried out. The first type is a simple developmental screen in which wild-type embryos are exposed to small molecules from a chemical library, and small molecules that induce specific developmental defects are identified. Screens of this type have led to the discovery of dozens of compounds that cause specific defects in hematopoiesis, cardiac physiology, embryonic patterning, pigmentation, and morphogenesis of the heart, brain, ear, and eye (Moon et al., J. Am. Chem. Soc. 124:11608-11609, 2002; Khersonsky et al., J. Am. Chem. Soc. 125:11804-11805, 2003; Peterson et al., Proc. Natl. Acad. Sci. U.S.A. 97:12965-12969, 2000; Peterson et al., Current Biology 11:1481-1491, 2001; Spring et al., J. Am. Chem. Soc. 124:1354-1363, 2002; Sternson et al., J. Am. Chem. Soc. 123:1740-1747, 2001). Many of the compounds discovered appear to be quite specific, with phenotypes comparable to those caused by specific genetic mutations, and some of the compounds are potent, with EC50s in the low nanomolar range (Peterson et al., Current Biology 11:1481-1491, 2001).

A second type of zebrafish small molecule screen is the modifier screen in which small molecules capable of modifying a disease phenotype are identified. We recently demonstrated the feasibility of this approach by identifying a novel class of compounds capable of suppressing the gridlock mutation (Peterson et al., Nat. Biotechnol. 22:595-599, 2004). Zebrafish gridlock mutants exhibit a dysmorphogenesis of the aorta that prevents circulation to the trunk and tail and is considered to be a model of human coarctation of the aorta (Weinstein et al., Nat. Med. 1:1143-1147, 1995). Gridlock mutants were exposed to 5,000 compounds from a diverse small molecule library. Two structurally related compounds were identified that completely restore gridlock mutants to normal without causing additional developmental defects (Peterson et al., Nat. Biotechnol. 22:595-599, 2004). Beyond their ability to suppress the gridlock phenotype in zebrafish, the gridlock suppressor compounds promote tubulogenesis in cultured human endothelial cells, showing that the compounds may be vasculogenic in fish and in mammals (Peterson et al., Nat. Biotechnol. 22:595-599, 2004). This finding is consistent with the observation that many drugs have similar activities in zebrafish and humans (Langheinrich, Bioessays 25:904-912, 2003; Milan et al., Circulation 107:1355-1358, 2003). Therefore, compounds that suppress disease phenotypes in zebrafish may have direct utility as lead compounds for human therapies. Zebrafish models of leukemia have been generated by ectopic expression of genes that have demonstrated roles in the pathogenesis of human leukemias (see, e.g., Langenau et al., Science 299:877-890, 2003; Kalev-Zylinska et al., Development 129:2015-2030, 2002). These models are not practical for use in high-throughput screening methods, however, due to reasons ranging from variable latency of tumor development, the high mortality rate of fish with germline transmission, transiency of expression, and difficulty in control of expression.

The personal and societal burden of AML is high. In the United States alone, about 7,000 people die each year from AML. The remarkable success of targeted therapies for chronic myeloid leukemia and acute promyelocytic leukemia are among the most encouraging successes in cancer treatment (Sawyers, Nature 432:294-297, 2004; Chabner et al., Nat. Rev. Cancer 5:65-72, 2005; Tallman, Semin. Hematol. 41:27-32, 2004). The benefit of the development of targeted therapies of AML would thus be very significant.

### SUMMARY OF THE INVENTION

We have generated a stable transgenic zebrafish line that expresses AML1-ETO from an inducible promoter. Adults from this line can be used to generate tens of thousands of transgenic zebrafish embryos at a time. Induction of the expression of the transgene causes a reproducible AML surrogate phenotype that can be readily detected in the intact zebrafish embryo within two days of fertilization. This line can be used in high-throughput assays for identifying small molecule suppressors of AML.

Accordingly, the invention provides methods for identifying agents (e.g., small organic molecules) that can be used in the treatment of acute myelogenous leukemia (AML). These methods involve: (i) providing a zebrafish that expresses (e.g., stably expresses) a gene product (e.g., a protein, such as a fusion protein including sequences of AML1 (e.g., the DNA binding domain of AML1) and ETO (e.g., human AML1 and ETO)) that induces a phenotype characteristic of AML (e.g., a gene product that blocks myeloid differentiation in AML), optionally, under the control of an inducible promoter (e.g., a heat shock protein (e.g., hsp70) promoter), (ii) inducing expression of the gene product (when an inducible promoter is used), (iii) contacting the zebrafish with a candidate agent, and (iv) analyzing the effect of the agent on an AML-related phenotype of the zebrafish.

Detection of an improvement in one or more AML-related phenotypes in the zebrafish, in the presence of a candidate agent, indicates the identification of an agent that can be used in the treatment of AML, or tested in additional model systems for such treatment. The phenotype analyzed can be, for example, loss of circulation, accumulation of hematopoietic cells in the intermediate cell mass (ICM), and/or loss of hematopoietic cell maturation as detected by analysis of a hematopoietic marker (e.g., PU.1, GATA-1, myeloid-specific peroxidase (MPO), or SCL), as can be caused by AML1-ETO. Preferably, the zebrafish subject to these tests are embryos, as described elsewhere herein. Expression of the gene product can be induced, for example, at 4-12 (e.g., 4, 16, or 24) hours post fertilization, and the phenotype can be monitored, for example, at 24-72 (e.g., 24, 48, or 72) hours post fertilization. The improvement detected in these methods can be, for example, an increase in circulation, a decrease in accumulation of hematopoietic cells in the ICM, and/or an increase in hematopoietic cell maturation, as detected by analysis of a hematopoietic marker (e.g., PU.1, GATA-1, myeloid-specific peroxidase (MPO), or SCL).

In preferred examples, the methods of the invention involve analysis of multiple zebrafish, which are present in separate wells of a multi-well plate, and are contacted with different candidate agents. Further, in these examples, an automated system can advantageously be used to monitor the phenotypes of the zebrafish, as described elsewhere herein.

The invention also provides zebrafish (mature or embryos) that include (e.g., stably express) a gene encoding a gene product that induces a phenotype characteristic of AML, optionally under the control of an inducible promoter. As an example, the gene product can be an AML1-ETO fusion protein (e.g., a human AML1-ETO fusion protein), optionally under the control of an inducible promoter (e.g., a heat shock protein promoter, such as that of hsp70). Such a fusion protein can include the DNA binding domain of AML1. Other examples of fusion proteins that can be expressed in the zebrafish of the invention are provided below.

Further, the invention provides methods of identifying therapeutic agents, which involve: (i) providing a zebrafish exhibiting a phenotype characteristic of a disease or condition, (ii) incubating the zebrafish in the presence of a candidate therapeutic agent, and (iii) monitoring the phenotype of the zebrafish using an automated system. In these methods, detection of an improvement in the phenotype indicates the identification of a therapeutic agent that can be used in the treatment of the disease or condition. The phenotype characteristic of the disease or condition can be due to, for example, a mutation in the zebrafish or induction of expression of a transgene encoding a protein that causes the phenotype characteristic of the disease or condition.

The invention also includes methods of treating AML by increasing TIS11b levels and/or activity in patients. TIS11b itself, a nucleic acid molecule encoding TIS11b, or a compound that activates expression, increases stability, and/or increases activity of TIS11b can be administered to patients, according to the invention.

Also, the invention includes methods for identifying agents that can be used in the treatment of AML. In these methods, a candidate agent is introduced into an expression system (e.g., a cell) that includes a gene encoding TIS11b. Then, the effect of the candidate agent on expression, stability, and/or activity of TIS11b is determined.

The invention provides several advantages. For example, the zebrafish models of the invention are characterized by an AML phenotype that is easily detected and monitored as the animals are contacted with candidate therapeutic compounds. Because of their permeability, the zebrafish model system of the invention is well-suited for use in chemical genetic screens, as described herein, which are powerful approaches to identifying physiologically relevant agents.

Further, the invention facilitates screening in a physiologically relevant context, allowing testing for efficacy and lack of toxicity in a whole, vertebrate animal, which cannot be achieved with in vitro or cell-based assays, and conveniently combines lead discovery and early animal testing into one step. In addition, the screening methods of the invention are not limited to a single target but, rather by targeting the AML phenotype in general, targets the full complement of potential molecular targets, possibly through one or more novel mechanisms. Even with the benefits provided with whole organism screening, as discussed above, such organisms are not generally amenable to assays involving high-throughput and automation. Zebrafish make it possible to combine the physiological context of the whole organism with high-throughput screening, and when used in the context of the present invention, provides small molecule screens to be performed to identify compounds that specifically reverse the effects of AML1-ETO expression.

Other features and advantages of the invention will be apparent from the following detailed description, the drawings, and the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Expression of AML1-ETO in zebrafish embryos causes a reproducible accumulation of hematopoietic blast cells in the intermediate cell mass (ICM). a) The DNA fragment containing 4 kb of zebrafish Hsp70 promoter and the human AML1-ETO fusion gene was used to generate the transgenic zebrafish line Tg(hsp:AML1-ETO). b) While wild-type embryos exhibit a robust circulation at 44 hpf, AML1-ETO-expressing embryos exhibit no circulating blood and an accumulation of hematopoietic cells in the ICM (arrowhead). c) Hematopoietic cells stained with diamino benzamidine are seen throughout the vasculature in wild-type embryos, but primarily in the ICM (arrowhead) of AML1-ETO-expressing embryos. d) Lack of circulation in AML1-ETO-expressing embryos is not caused by a vascular obstruction as evidenced by microangiography. e-k) Accumulation of immature hematopoietic blast cells in Tg(hsp:AML1-ETO) zebrafish embryos. Cytology of blood cells collected from the ICM of wild-type (e, h-i) or Tg(hsp:AML1-ETO) (f-g, j-k) zebrafish embryos at 40 hpf. All of the embryos have been subjected to four 1-hour 37° C. heat treatments at 12-hour intervals. e-g) The ICM collections contain predominantly mature erythrocytes which are nucleated in zebrafish. However, cells with immature blast-like morphology can be identified from the transgenic collections (g, arrowheads). h-k) Clusters of blood cells from wild-type and transgenic embryos are shown at higher magnification. h, i) Cell clusters from the control embryos are composed predominantly of mature erythrocytes, while other myeloid cell types such as mature, bi- or tri-nucleated heterophils/neutrophils (h, arrow) can be found only occasionally. However, clusters of large blast-like early cells can be readily identified in samples from the transgenic animals (j, k).

FIG. 2. The blood accumulation phenotype in Tg(hsp:AML1-ETO) zebrafish embryos is dependent on AML1-ETO expression. a) Injection of hAML1-MO rescues the blood accumulation phenotype in heat-treated transgenic animal. Arrows point to the accumulated blood cells. b) Injection of solution containing 500 μM HAML1-MO significantly decreases the percentage of AML1-ETO-expressing embryos without circulating blood to 6.7% compared to 90.2% of the non-injected control. c) The Tg(hsp:AML1-ETO) embryos were incubated at 42° C. for one hour to induce AML1-ETO expression from 16 to 24 hpf as indicated in the graph. The embryos were heat-treated again 6 hours after their first heat shock to maintain transgene expression. The percentage of embryos exhibiting the loss-of-circulation phenotype was scored at 40 hpf. WT, wild-type; TG, transgenic; HS, heat shock.

FIG. 3. Retinoic acid partially rescues the AML1-ETO phenotype in zebrafish embryos. Tg(hsp:AML1-ETO) embryos were subjected to a total of three 1-hour 37° C. heat treatments at 4, 16, and 24 hpf. All-trans retinoic acid (1 pM to 1 nM) or the vehicle (DMSO) was added into the fish water before the final heat treatment at 24 hpf. The percentage of embryos with circulation was scored at 40 hpf as an indication of rescue.

FIG. 4. The transcriptional changes in the hematopoietic cells of AML1-ETO-expressing zebrafish embryos. Blood samples were obtained from wild-type and Tg(hsp:AML1-ETO) embryos that had both been subjected to three 1-hour heat treatments at 12-hour intervals. cDNAs synthesized from the transcripts of the blood samples were then used for real-time PCR. The fold of expression of each gene was obtained by comparing transcript quantity in the transgenic samples to the quantity in wild-type samples after each had been normalized to GAPDH levels in these samples. The graph represents the mean ratio±the SEM.

FIG. 5. Blockage of TIS11b expression enhances the AML1-ETO phenotype. a) Under a mild heat shock condition, even though most of the non-injected Tg(hsp:AML1-ETO) embryos still exhibit circulating blood, almost all of the transgenic embryos injected with zebrafish TIS11b antisense morpholino oligonucleotide (zTIS11b-MO) show the accumulation of hematopoietic cells in the ICM. zTIS11b-MO did not block circulation in wild-type embryos under the same conditions. Arrowheads point to where significant amounts of blood are seen. b) Injection of solution containing 200 μM zTIS11b-MO enhances the AML1-ETO phenotype. The percentage of fish embryos without circulating blood is used as the indication of AML1-ETO phenotype. c) The blood extracted from the transgenic embryos injected with zTIS11b-MO contains abundant immature blast-like cells (arrows). WT, wild-type; TG, transgenic; HS, heat shock.

FIG. 6. The lack of circulation phenotype can be determined automatically by digital image subtraction. The top row is a wild-type embryo with circulation, whereas the bottom row is a transgenic embryo without circulation.

FIG. 7. A flowchart of the branching variable used to identify the locations of zebrafish within the wells of the 96-well assay plates is shown.

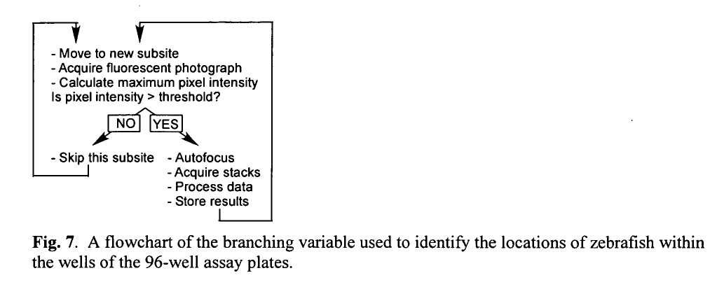

### DETAILED DESCRIPTION

As is discussed above, approximately 15% of all cases of acute myelogenous leukemia (AML; FAB-M2 subtype) are caused by a t(8;21) chromosomal translocation that results in fusion of AML1 and ETO proteins (Koeffler, Ann. Intern. Med. 107:748-758, 1987; Tashiro, Cancer 70:2809-2815, 1992). We have developed a model of AML in zebrafish using a transgenic line that stably expresses a human AML1-ETO fusion protein under the control of an inducible promoter. Induced AML1-ETO expression causes a block in hematopoietic maturation that manifests itself as a reproducible accumulation of immature hematopoietic progenitors in the intermediate cell mass (ICM) and a concomitant loss of circulating cells, and these phenotypes can be readily detected in the intact, transparent zebrafish. According to the invention, this model of AML can be used in automated, whole-organism, high-throughput assays to screen for small molecules that reverse the AML1-ETO phenotype.

The invention thus provides animal model systems for use in identifying agents that can be used to treat AML, high-throughput methods of using these systems to identify such agents, as well as methods of treating patients with the identified agents. As discussed elsewhere herein, the systems of the present invention are advantageous because, for example, in facilitating drug screens in an in vivo, physiologically relevant context, the likelihood that an agent identified in the system will be effective in another physiological context (e.g., a human patient) is increased. Further increasing the likelihood of identifying an effective agent, the screens of the invention focus on detecting correction of a phenotype that is characteristic of a disease, rather than being limited to a particular target. An additional advantage of the systems of the invention is that they enable high-throughput screening, greatly increasing the number of candidate agents that can be screened. The animal model systems of the invention, as well as the screening methods employing the systems, are described further, as follows.

Zebrafish System

As is discussed above, the zebrafish provides a powerful tool for phenotype-based screens, due to its optical transparency and external development, which make real time observation of its internal organs simple. Further, the optical clarity of the zebrafish embryo enables the use of fluorescent markers that highlight the locations or activities of specific populations of cells, which can greatly facilitate detection of anatomical changes caused by agents such as small molecules. Conveniently, during the embryonic and larval stages of life, the zebrafish is only about 1-2 mm long, and can live for days in a single well of a standard 384-well plate, surviving on nutrients stored in its yolk sac. These features make it possible to perform large-scale, phenotype-based screens. Further, because screening can be performed in the whole organism, perturbation of potential therapeutic targets by agents such as small molecules reveals the effects of such perturbations on the integrated physiology of the entire organism. In addition, the unique attributes of the zebrafish embryo allow ‘chemical genetic’ technologies to be applied to complex diseases such as leukemia, as zebrafish embryos readily absorb small molecules from the surrounding medium. In the current invention, the zebrafish small molecule screen is the modifier-type screen (see above), in which small molecules capable of modifying a disease phenotype are identified.

We have generated transgenic zebrafish that stably express a human AML1-ETO fusion protein from an inducible promoter. Adults from this line can be used to generate tens of thousands of transgenic zebrafish embryos at a time. Induction of the transgene causes a reproducible AML surrogate phenotype that can be readily detected in the intact zebrafish embryo within two days of fertilization. Advantageously, expression of the transgene is controlled by an inducible promoter, so that expression can be induced at an appropriate time (for example, 4-24 (e.g., 4, 16, or 24) hours post fertilization (hpf)). This is important, as expression of the fusion protein earlier in development may result in lethality.

Any inducible promoter can be used in the invention, as determined to be appropriate by those of skill in the art. As discussed below, one type of inducible promoter that can be used in the invention is the zebrafish hsp70 heat shock protein promoter. Stable expression of a construct including this promoter, as well as methods for inducing expression from the promoter, are discussed further below in the experimental examples. Additional examples of inducible promoters that can be used in the invention include heat/laser inducible systems (Halloran et al., Development 127(9):1953, 2000), promoters induced or inhibited by doxycycline/tetracycline and their derivatives, inducible systems involving RU486 and its derivatives, and inducible systems involving use of the metallothionein promoter.

A specific example of an AML1-ETO fusion protein that can be used in the invention is described below in the experimental examples (also see, e.g., Kalev-Zylinska et al., Development 129:2015-2030, 2002). In addition to this particular fusion protein, other AML1-ETO fusion proteins that occur in AML (e.g., human AML) or lead to a similar phenotype in zebrafish can be used in the invention. As examples, proteins that include additional AML1 sequences, fusion proteins that are truncated on one or both ends, proteins in which fusions occur at differing locations, or fusion proteins including mutations as compared to wild type sequences can be used. In general, the fusion proteins include the DNA binding domain of AML1 (e.g., amino acids 1-177 of human AML1) and the complete sequence of ETO. Alternatively, additional AML1 sequences can be included, or a truncated or mutant AML1 sequence can be used, which preferably maintains DNA binding capability. The sequence of ETO can also be truncated or mutated but, if so, it preferably maintains the ability to recruit the nuclear receptor co-repressor (N-CoR)/mSin3/histone deacetylase (HDAC) complex, as the AML1-ETO fusion product is thought to act by repressing the transcription of the genes that are normally activated by the CBF complex (see above). Determining whether a candidate fusion protein can be used in the invention is straightforward, as the fusion protein can be expressed in zebrafish, which are then analyzed for one or more of the phenotypes characteristic of AML, as described elsewhere herein.

In addition to the AML1-ETO fusion protein described above, any other translocation products associated with AML can be used in the animal model systems of the invention (see, e.g., Scandura et al., Oncogene 21:3422-3444, 2002). For example, any of the following fusions can be used: AML1/ETO (e.g., t(8;21)(q22;q22)), AML1/MTG16 (e.g., t(16;21)(q24;q22)), AML1/EV11 (e.g., t(3;21)(q26;q22)), CFBβ/MYH11 (e.g., Inv(16)(p13;q22), or t(16;16)(p13;q22); also, CFBβ del(16)(q22)), PML/RARα (e.g., t(15;17)(q22;q12)), PLZF/RARα (e.g., t(11;17)(q23;q12)), NPM/RARα (e.g., t(5;17)(q35;q12)), NuMA RARα (e.g., t(11;17)(q13;q12)), STAT5b/RARα (e.g., t(17;17)(q11;q12)), MLL/AF4 (e.g., t(4;11)(q21;q23)), MLL/AF6 (e.g., t(6;11)(q27;q23)), MLL/AF9 (e.g., t(9;11)(p22;q23)), MLL/ENL (e.g., t(11;19)(q23;p13;3)), MLL/ELL (e.g., t(11;19)(q26;p13.1)), MLL/EEN (e.g., t(11;19)(q23;p13.3)), MLL/CBP (e.g., t(11;16)(q23;p13)), MLL/p300 (e.g., t(11;22)(q23;q13)), NUP98/HOXA9 (e.g., t(7;11)(p15;p15)), NUP98/HOXD13 (e.g., t(2;11)(q31;p15)), NUP98/PMX1 (e.g., t(1;11)(q24;p15)), NUP98/DDX10 (e.g., inv(11)(p15;q22)), DEK/CAN (e.g., t(6;9)(p23;q34)), MOZ/CBP (e.g., t(8;16)(p11;p13)), BCR/ABL (e.g., t(9;22)(q34;q11)), and TLS/ERG (e.g., t(16;21)(p11;q22)). Further, the model systems can be characterized by overexpression of EVI-1 (e.g., t(3;3)(q21;q26) or inv(3)(q21;q26)) or p53 mutations (e.g., del(17p)). (See, e.g., Mrozek et al., J. Clin. Oncol. 19(9):2482-2492, 2001, for additional examples and information concerning translocations and mutations.)

Zebrafish for use in the invention can be made using standard methods. For example, a linearized construct including a gene encoding a translocation product characteristic of AML, such as an AML1-ETO fusion protein, as described herein, or any other fusion protein associated with AML (see above), under the control of an inducible promoter (see above), can be injected into 1 cell zebrafish embryos. Zebrafish carrying the transgene are then identified by, for example, genotyping involving PCR analysis of fin-clips.

Screening Method

The screening methods of the invention, which involve the identification of suppressors (e.g., small molecules) of the zebrafish AML1-ETO phenotype, can involve visual inspection of AML1-ETO zebrafish embryos to determine the presence or absence of the AML1-ETO phenotype. As is discussed elsewhere herein, this phenotype can be detected by observation of, for example, a lack of circulation, accumulation of cells in the ICM, and/or loss of expression of hematopoietic markers. In these methods, expression of AML1-ETO is induced (at, e.g., any one or more time points between 4 and 40 hpf), zebrafish are incubated in the presence of one or more candidate compounds (at, e.g., 18-24 hpf), and the effects of the compounds on one or more AML1-ETO phenotypes is assessed (at, for example, 24-72, e.g., 40-48 hpf). The time frames noted above are exemplary only because, due to the flexibility of the system, earlier and later time points can be used as well.

Candidate compounds that can be tested in the invention can come from many different sources including, for example, large libraries of natural products, synthetic (or semi-synthetic) extracts, and chemical libraries. Those skilled in the field of drug discovery and development will understand that the precise source of test compounds or extracts is not critical to the methods of the invention. Candidate compounds to be tested include purified (or substantially purified) molecules or one or more components of a mixture of compounds (e.g., an extract or supernatant obtained from cells) and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including MicroSource Discovery Systems (Gaylordsville, Conn., U.S.A.), Biotics (Sussex, UK), Xenova (Slough, UK), Harbor Branch Oceanographic Institute (Ft. Pierce, Fla., U.S.A.), and PharmaMar, U.S.A. (Cambridge, Mass., U.S.A.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H., U.S.A.) and Aldrich Chemicals (Milwaukee, Wis., U.S.A.). Libraries of synthetic compounds can also be produced, if desired, according to methods known in the art, e.g., by standard extraction and fractionation. Furthermore, if desired, any library or compound can be readily modified using standard chemical, physical, or biochemical methods.

When a crude extract is found to have an effect on an AML-related phenotype, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

Visual inspection of zebrafish for the effects of candidate compounds on phenotypes characteristic of AML, such as AML1-ETO-related phenotypes, permits about 400 small molecules to be screened per hour, requires significant concentration and effort, and is subject to the opinion of the individual screener. High-throughput, automated approaches, as described herein, can increase the efficiency of such assays and eliminate subjectivity. These types of assays are generally described further, as follows, with AML1-ETO as an exemplary transgene. Although specific examples and values are noted below for many parameters of the assays, the materials and values used can be varied, as understood by those of skill in the art. A more specific example is provided in the experimental section, below.

Automating the Circulation Assay (Primary Screen)

Embryo Generation and Handling

Embryos can be generated by mating homozygous Tg(hsp:AML1-ETO) adults with homozygous transgenic fish that express a detectable product, such as GFP, in hematopoietic cells under control of, for example, the GATA-1 promoter (Tg(gata1:GFP); Long et al., Development 124:4105-4111, 1997). As is described below, although it is possible to perform these experiments without the use of the Tg(gata1:GFP) line, the fluorescent hematopoietic cells can increase assay sensitivity, facilitate autofocusing and object finding, and increase the throughput of the assay. The embryos are subjected to heat shock in bulk at 4-24 (e.g., 4, 16, or 24) hpf as described below. This heat shock regimen produces the AML1-ETO phenotypes of hematopoietic cell accumulation in the ICM and lack of circulation in 94% of embryos. After the 24 hpf heat shock, embryos are distributed 3 embryos per well into the wells of opaque black 96-well plates with flat transparent bottoms (Corning Costar). Embryo distribution can be performed manually using a glass pipette but, advantageously with respect to high-throughput methods, as described herein, can be performed automatically by an embryo sorter, such as a COPAS XL embryo sorter (Union Biometrica). The assay plates containing embryos can then be incubated at 28.5° C. until 48 hpf, at which time they can be imaged for analysis.

Automating Object Finding

Individual zebrafish can be identified in the wells of the 96-well plates using, for example, maximum intensity measurements and a branching variable. An automated microscope can systematically examine each well by querying 4 non-overlapping virtual sub-sites for the presence of a fluorescent object (GFP-positive hematopoietic cells). At each sub-site, a fluorescent image is acquired and the maximum pixel intensity is measured. When an embryo is present, the maximum pixel intensity is significantly higher than background. A branching variable based on maximum pixel intensity is used to identify sub-sites with fluorescent objects (embryos). If the maximum pixel intensity is above an empirically determined threshold, an embryo is present, while if the maximum pixel intensity is below the threshold, no embryos are present. If an object is not present, the next sub-site is queried. If an object is present, a series of additional tasks is performed, including autofocus and automated imaging of the embryos as described below.

Optimizing Autofocus

As described below in the experimental results section, circulation was easily detected by digital image subtraction once the focal plane was set manually. For automated screening, focusing can be performed automatically. For example, the MetaMorph autofocus function can be used to focus on the fluorescent hematopoietic cells in the embryo (Long et al., Development 124:4105-4111, 1997). Autofocusing can be achieved using a piezo focus motor (Physik Instrumente) to control objective height under control of the MetaMorph software. Images are captured beginning at a prespecified Z origin and at successive Z positions within a prespecified range. Image sharpness at the brightest spot is measured for each Z position, and the Z position is adjusted with successive iterations until focus meeting the specified level of accuracy is achieved. Optimization of the following values can be carried out: Z origin, maximum step size, maximum number of Z moves, autofocus range, and required degree of focus accuracy. The optimal values for each of these factors can be determined by trial and error using a 96-well plate containing three 48 hpf Tg(gata1:GFP) embryos in each well. A value can be considered to be optimized if it allows the autofocus operation to be completed in the minimum amount of time without causing the detection rate to fall below 95% (i.e., automated detection of circulation in 95% of the embryos).

Optimizing Stack Frame Number

The automated detection of circulation described below in the experimental results section was performed by acquiring 2 consecutive 20-frame image stacks and subtracting one stack from the other. The differences between each pair of frames can be added to increase the signal strength. The resultant image is the summed differences image and produces a robust signal from circulating blood cells. Although it is possible to perform the screen using this method, it is also possible to reduce the number of frames required to detect circulation, especially given the signal enhancement obtained by imaging the fluorescent hematopoietic cells of the Tg(gata1:GFP) embryos. The optimal number of frames to capture can be determined by testing all possible frame numbers from 1 to 20 using a 96-well plate containing three 48 hpf Tg(gata1:GFP) embryos in each well. The optimal frame number is the lowest number that allows circulation to be detected without causing the detection rate to fall below 95% (i.e., automated detection of circulation in 95% of the embryos).

Optimizing Data Processing—Digital Subtraction, Thresholding, and Object Filtering

Once two consecutive image stacks have been acquired, movement is detected by subtracting each image from stack 1 from the corresponding image from stack 2, and then summing the results from each pair of subtracted frames to generate the summed differences image. Where there is no circulation, there are no changes from one image to another, resulting in a blank image. Circulating cells produce differences between frames. After subtraction, the path of circulation appears as an object surrounded by a blank background. The object representing the path of circulating cells can be identified and analyzed further by MetaMorph. The object is identified using a thresholding algorithm that identifies objects with signal intensities within a specified range. Noise and artifacts are removed by filtering objects to include only those that are of the approximate size and shape of the zebrafish vasculature. The MetaMorph software can be programmed to perform all of these functions sequentially-object finding, autofocusing, capture of stacks 1 and 2, digital subtraction, generation of the summed differences image, thresholding, and object filtering—for each well of a 96-well plate. MetaMorph can be programmed to save all of the summed differences images and to output a list of wells in which circulating hematopoietic cells are present.

The time required to carry out each step of the described here is summarized in the following table.

Time required using current settings, 3 embryos/well

| Process | Time per step current (target) | Current time/well | Target time/well |
|---|---|---|---|
| Object finding | 4 quadrants × 50 msec = | 200 msec = | 200 msec |
| Autofocusing | 3000 (2000) msec/embryo = | 9000 msec = | 6000 msec |
| Stack acquisition | 2 stacks × 20 (10) frames × 50 msec/embryo = | 6000 msec = | 3000 msec |
| Stage movement = | | 800 msec max. = | 800 msec |
| Data processing | 2000 msec concurrent with acquisition = | 16000 msec/well = | 10000 msec/well |

Scoring as positive only those wells in which all three embryos have restored circulation can be used as an approach to manage the possibility of false positives. The heat shock protocol described herein produces the AML1-ETO phenotypes of hematopoietic cell accumulation in the ICM and lack of circulation in 94% of embryos. The probability that all three embryos in a well exhibit circulation because of incomplete penetrance of the phenotype is 0.06³=2×10⁻⁴. Although the rate of true positives in a zebrafish screen varies from assay to assay, the typical range is from 0.0004 to 0.01 (Peterson et al., Proc. Natl. Acad. Sci. U.S.A. 97:12965-12969, 2000; Peterson et al., Nat. Biotech. 22:595-599, 2004). Thus, true positives are expected to outnumber false positives significantly.

In the event that embryo orientation presents problems with respect to detection of circulation, as may be the case at 48 hpf, use of the Tg(gata1:GFP) line will help eliminate this problem. In particular, because the embryo is transparent, it should be possible to focus on and image the fluorescent blood cells from virtually any orientation. In our automated heart rate assay, we found that heart motion could be detected in embryos in every orientation, and we expect that the motion of circulating blood cells will be easier to detect. Otherwise, image acquisition can be performed at 72 hpf, when embryos are hatched and typically adopt a more uniform, extended orientation along the bottom of the well.

Further, zebrafish embryos exhibit occasional spontaneous movements beginning 17 hpf (Saint-Armant et al., J. Neurobiol. 37:622-632, 1998), and such movement during image acquisition could result in strong signals in the summed differences image due to significant differences between frames being subtracted. In most cases, the spontaneous movements will be much larger than the movement associated with circulation and will therefore be easy to filter out during the object filtering step. However, small amplitude spontaneous movements could possibly cause a ‘false positive.’ At 48 hpf, large amplitude spontaneous movements occur approximately once every 2-3 minutes, and small amplitude movements are much less frequent. We therefore expect the probability of one false positive in a well to be <10², and the probability of all three embryos to be undergoing small amplitude spontaneous movements during acquisition to be <10⁻⁶. If, however, it is found that the false positive rate is higher than this due to spontaneous movement, tricaine (0.006%) can be added to the embryo buffer. This compound anesthetizes the embryos and eliminates spontaneous movements without disrupting heart function.

Because the assay described herein is more complex and content rich than most in vitro assays, screening is slower. Using the current settings described above and a single screening instrument, screening 100,000 compounds would require 16 seconds/well or 444 hours of screening. By optimizing autofocusing and stack frame number, the screening speed can be increased enough to perform the screen in 10 seconds/well or a total of 278 hours. By using round-bottom or v-bottom wells, all 3 embryos would be forced into a single quadrant. This eliminates the need to perform object finding and allows information concerning all three embryos to be acquired simultaneously. This reduces the screening time by a further factor of 3, to approximately 90 hours of screening. Finally, the assay can be multiplexed by adding 5 compounds to a single well. This requires a deconvolution step to determine the identities of any hits, but further decreases the screening time by a factor of 5. Therefore, despite the complexity of the assay, a combination of these solutions enables truly large-scale screens to be performed using a single instrument in 2-3 days.

It is possible that a small molecule may rescue the hematopoietic defect caused by expression of AML1-ETO, but not be detected because it also causes a developmental defect (e.g., a cardiovascular defect) that prevents restoration of circulation. This requirement of low toxicity is one of the advantages of using a whole organism—a compound that rescues the defect without causing other toxicities to the organism may be more useful than one that only has in vitro activity. However, it is also helpful to identify compounds that suppress the hematopoietic defect in addition to causing other toxicities. Such compounds may cause a detectable change in the expression of markers of mature hematopoietic cells, and can be identified using, for example, a secondary screen such as that described further below.

Testing the Sensitivity, Specificity, and Reproducibility of the Circulation Assay (Primary Screen)

The sensitivity, specificity, and reproducibility of the fully-automated circulation assay of the invention can be tested by analysis of 96-well plates filled with embryos displaying the AML1-ETO phenotype, the wild-type phenotype, or intermediate phenotypes. In particular, in one example, Tg(hsp:AML1-ETO) zebrafish are mated with Tg(gata1:GFP) zebrafish to produce doubly transgenic embryos. Half of the embryos are subjected to the standard heat shock regimen described above. Embryos are distributed three embryos per well into three 96-well plates as follows: one plate of heat shocked embryos, one plate of unshocked embryos, and one plate of heat shocked embryos in which two wells have been replaced with unshocked embryos. At 48 hpf, the three plates are scored for circulation visually using a dissecting microscope and using the automated screening system described in the previous section. The rates of false positives and false negatives are calculated as the percentages of correlation between results from visual and automated detection of circulation.

As described previously, it is expected that approximately 6% of embryos in the heat-shocked plate will exhibit circulation due to the incompletely penetrant phenotype. In addition, we expect up to 1% of embryos from this plate to score as false positives due to spontaneous embryo movement. In total, we expect 7% of individual embryos to score as positive, but expect 0.03% (0.07³) of wells to meet the requirements of a positive (3/3 embryos with circulation). Significantly higher numbers of embryos scoring as positive will indicate additional sources of false positives that need to be minimized. All embryos in the wild type plate should possess circulations. We expect that the rate of detection will approach 100% for this plate, but the empirical value obtained indicates the percentage of true positives that are likely to be detected. The mixed plate confirms the ability of the automated assay to pick out true positives among a background of negatives. We expect that the rate of false positives will be comparable to that determined for the heat shocked plate.

False positives are likely to be more common in this assay than false negatives. Our preliminary results suggest that up to 7% of the individual embryos will be scored as having circulation (6% due to incomplete penetrance of phenotype and 1% due to motion artifacts), and that this percentage will result in a low overall false positive rate. If the number of embryos with circulation increases above 10%, it would begin to make the assay unfeasible. However, by including three embryos in each well, we effectively are performing the assay in triplicate, and even a 10% false positive rate at the embryo level results in an overall assay false positive rate of 0.1³=0.001. In a screen of 100,000 small molecules, this would lead to the identification of 100 false positives, which could easily be eliminated by retesting of those 100 compounds. If the false positive rate is higher than this threshold, the stringency of the heat shock protocol can be increased and the image acquisition parameters and data processing algorithms adjusted to reduce the false positive rate below 10% of individual embryos. A high percentage of false negatives is unexpected and less problematic for the assay. If less than 90% of the wild-type embryos are identified as having circulation, the data processing parameters can be adjusted to make the assay more sensitive. For example, the threshold parameters can be decreased so that less motion is detected, and the range of tolerated object sizes can be expanded in the object filtering step.

Development of an Assay for Detection of Hematopoietic Maturation (Secondary Screen)

In the primary assay described above, lack of circulation is a surrogate phenotype that is a readily-detectable reflection of AML1-ETO activity. Perturbations that restore circulation to AML1-ETO expressing fish likely do so by influencing AML1-ETO or its critical downstream effectors. However, it is also useful to have a quantitative secondary assay that confirms the specificity of any hits and aids in determining the mechanism of rescue. An assay that measures the degree of maturation of hematopoietic precursors is particularly useful in this regard.

In humans, mutations in PU.1 are associated with AML (Mueller et al., Blood 100:998-1007, 2002) and AML1-ETO physically binds and inactivates PU.1 (Vangala et al., Blood 101:270-277, 2003). Overexpression of PU.1 promotes differentiation of AML1-ETO-expressing Kasumi-1cells to the monocytic lineage (Vangala et al., Blood 101:270-277, 2003). Therefore, PU.1 expression level is a useful measure of hematopoietic maturation. We have shown using quantitative PCR that in our zebrafish model of AML, expression of the myeloid master regulator PU.1 is reduced reproducibly to less than half the quantity detected in wild-type embryos. The promoter elements that regulate expression of PU.1 in zebrafish have been characterized and used for the generation of a transgenic zebrafish reporter line (Hsu et al., Blood 104:1291-1297, 2004). The zebrafish PU.1 promoter can be used to generate a reporter strain, such as a luciferase-based zebrafish reporter strain, which provides a quantitative, in vivo readout of hematopoietic maturation. This secondary assay can be used to confirm the specificity of any hits from the primary screen, but it can also be integrated with the primary assay and be performed in parallel as a high-throughput screen, or performed in the absence of the primary screen.

The promoter region that was previously used for tissue-specific expression of GFP (Hsu et al., Blood 104:1291-1297, 2004) can also be used to generate a quantitative transgenic reporter line. The sequence encoding GFP can be excised from the plasmid 5pu. 1-GFP (Hsu et al., Blood 104:1291-1297, 2004) and replaced with the sequence encoding firefly luciferase. The new plasmid, 5pu. 1-luciferase, is then used to generate a novel zebrafish line by injecting linearized plasmid into zebrafish embryos of the one cell stage as described (Grabher et al., Methods Cell Biol. 77:381-401, 2004; Udvadia et al., Dev. Biol. 256:1-17, 2003). Injected embryos are then raised to adulthood and tested by PCR (from fin clips) for transgenesis. Germline incorporation is confirmed by mating candidate transgenic carriers, lysing offspring, and subjecting the lysates to the Luciferase Reporter Assay (Promega). Once founders are identified with germline transmission of the transgene, the transgenic lines are bred to homozygosity. This transgenic reporter line can be referred to as Tg(pu.1:luc).

After a PU.1:luciferase reporter line for hematopoietic maturation is generated, the suitability of the assay can be tested for secondary confirmation of preliminary hits and for potential use in high-throughput screening. The assay can be tested by filling 96-well plates with embryos displaying the AML1-ETO phenotype, the wild-type phenotype, or intermediate phenotypes, and analysis of its ability to identify the hematopoietic maturation status of embryos in these plates.

Tg(hsp:AML1-ETO) zebrafish are mated with the transgenic luciferase reporter line Tg(pu.1:luc) to produce doubly transgenic embryos. Half of the embryos are subjected to the standard heat shock regimen described above. Embryos are distributed three embryos per well into three 96-well plates as follows: one plate of heat shocked embryos, one plate of unshocked embryos, and one plate of heat shocked embryos in which two wells have been replaced with unshocked embryos. At 48 hpf, the three plates are scored for hematopoietic maturation by lysing the embryos by addition of 25 μL of 5× Passive Lysis Buffer (Promega) to the 100 μL of fish water surrounding the embyros, followed by sonication. Luciferase activity is measured by transferring 20 μL of the lysates to clean 96-well assay plates and performing the Luciferase Reporter Assay (Promega) using a Wallac multiwell luminometer fitted with autoinjector. A threshold value is established that best differentiates wild-type from AML1-ETO-expressing samples. Ideally, this is at least three standard deviations above the average reading for the AML1-ETO-expressing plate. The rates of false positives will be equal to the percentage of AML1-ETO expressing wells with luciferase values above the threshold, and false negatives will be calculated as the percentages of wild-type wells with values below the threshold.

Promoters other than the PU.1 promoter can also be used for the generation of reporter lines including, for example, the gata-1, c-myb, and hbbe3 promoters. Sensitivity can be increased, as needed, by increasing embryo number per well, reducing the lysis volume, or by switching to a fluorescent protein reporter such as EGFP. As an alternative option to generating a transgenic line as a reporter for hematopoietic maturation using any of the promoters described, marker expression by quantitative PCR and/or whole mount in situ hybridization can be carried out.

Performing a Screen of 2000 Known Bioactive Compounds Using the Circulation Assay (Primary Screen) and the Hematopoietic Maturation Assay (Secondary Screen)

The following approach can be used to identify potential AML drugs, based on the circulation assay described above. In such an assay, it is possible to test any type of candidate compound. As an example, a library of known bioactives commercially available through MicroSource Discovery Systems (Gaylordsville, Conn., U.S.A.) can be tested. Approximately half of these compounds are pure natural products and their derivatives. They include simple and complex oxygen heterocycles, alkaloids, sequiterpenes, diterpenes, pentercyclic triterpenes, and sterols. The rest are synthetic compounds with biological activity. Three quarters of these compounds are FDA-approved. The library compounds are provided as 10 mM stock solutions dissolved in DMSO and have diverse biological activities including NMDA antagonists, urokinase inhibitors, phosphodiesterase inhibitors, aldol reductase inhibitors, adenosine receptor antagonists, PLA2 inhibitors, cholinesterase inhibitors, HT3 receptor agonists, lipoxygenase inhibitors, O-methyltransferase inhibitors, K-channel blockers, aminopeptidase inhibitors, NO synthase inhibitors, and many others. Other examples of compounds and types of compounds that can be screened include those discussed above.

This screen can be performed, for example, by mating 60 Tg(hsp:AML1-ETO) males with 60 Tg(gata1:GFP) females to generate more than 6,000 doubly transgenic embryos. Embryos are heat shocked following the standard protocol described above to induce expression of AML1-ETO. At 24 hpf, embryos are distributed three embryos per well into the wells of 96-well plates containing 250 μL of embryo buffer as described (Peterson et al., Methods Cell Biol. 76:569-591, 2004). One well in each plate is filled with three embryos that have not been heat shocked, as a positive control. Compounds from the known bioactives collection are added to the buffer surrounding the embryos using pin transfer of 100 nL from the stock solutions. The final compound concentration is 4 μM in each well. After addition of the small molecules to the plates, the embryos are incubated for an additional 24 hours, at which point they are analyzed for the presence of circulating hematopoietic cells using the automated circulation assay described above. Small molecules are scored as positives if they rescue the AML1-ETO phenotype in 3/3 embryos. Wells identified as containing three embryos with circulation are examined visually for confirmation, and initial positives are confirmed by retesting using a group of 50 transgenic embryos.

Many of the 2000 compounds used in such a screen may inhibit essential enzymes or perturb other critical biological pathways. Therefore, these compounds may cause general toxicity to the zebrafish embryo that could confound detection of AML-suppressive activity. These toxicities are mitigated by using 4 μM as the screening concentration and by adding the compounds at 24 hpf. We have screened a subset of these compounds (approximately 500 compounds) for their effects on zebrafish vascular development at various doses and treatment times. We have found that severe teratogenic effects are caused by many of these compounds when they are added prior to 24 hpf. However, only 2.5 percent of the compounds cause a detectable developmental defect when compounds are added at 16 hpf, possibly because many of the major developmental events are largely complete. In this screen, we can add the small molecules at 24 hpf, and the phenotype can be assessed at 48 hpf. Therefore, the small molecules have 24 hours to exert their effects prior to phenotypic assessment, and are less likely to cause confounding developmental defects. The screening concentration can be reduced further if toxicity appears to be confounding results.

The AML1-ETO hematopoietic maturation assay can also be used to identify small molecules with utility for targeted therapy of AML. For this screen, the same library of 2000 known bioactive small molecules described above for the circulation screen can also be used. These small molecules all possess biological activity, are structurally diverse, and target hundreds of distinct protein targets. Therefore, despite the relatively small scale of this screen, the likelihood of identifying small molecules that affect the AML1-ETO phenotype is increased. Beyond validation of the hematopoietic maturation assay per se, this screen can help cross-validate the primary (circulation) assay. A high degree of correlation between the results from the screens for the circulation and hematopoietic maturation assays suggest that the results are relevant and confirm the validity of the individual hits.

The screen can be performed by mating 60 Tg(hsp:AML1-ETO) males with 60 Tg(pu.1:luc) females that are homozygous carriers of the hematopoietic maturation reporter transgene to generate more than 6,000 doubly transgenic embryos. Embryos are heat shocked following the standard protocol described above to induce expression of AML1-ETO. At 24 hpf, embryos are distributed three embryos per well into the wells of 96-well plates containing 250 μL of embryo buffer as described (Peterson et al., Methods Cell Biol. 76:569-591, 2004). One well in each plate is filled with three embryos that have not been heat shocked as a positive control. Compounds from the known bioactives collection are added to the buffer surrounding the embryos using pin transfer of 100 nL from the stock solutions. The final compound concentration is 4 μM in each well. After addition of the small molecules to the plates, the embryos are incubated for an additional 24 hours, at which point they are lysed in high-throughput using Passive Lysis Buffer and sonication. The level of luciferase expression (as a surrogate for the degree of hematopoietic maturation) is determined by quantification using a Wallac luminometer fitted with an autoinjector. Small molecules are scored as positives if they induce a change in luciferase expression greater than three standard deviations from the mean obtained from a 96-well plate of untreated embryos. Wells identified as initial positives are confirmed by retesting using groups of 50 transgenic embryos.

Hits from the screens described herein and from large-scale screening that may follow are evaluated for their significance and prioritized for further study. The first step can involve testing the compounds in the following panel of zebrafish and mammalian AML assays, as well as additional animal model assays (e.g., mouse model assays).

i) zebrafish cytology. AML1-ETO-expressing zebrafish exhibit cytological defects reminiscent of human AML. Embyros are exposed to the test compound, blood is collected, and cytology is performed as described elsewhere herein. A decrease in the number of immature blast-like cells can be considered evidence of compound efficacy in this assay.

ii) zebrafish in situ hybridization. AML1-ETO-expressing zebrafish exhibit dramatically reduced expression of c-myb and hbbe3 by in situ hybridization (Kalev-Zylinska et al., Development 129:2015-2030, 2002). Embyros can be exposed to the test compound and processed for in situ hybridization using c-myb and hbbe3 as probes following standard protocols (Oxtoby et al., Nucleic Acids Res. 21:1087-1095, 1993). Increased expression of these markers can be considered evidence of hematopoietic maturation.

iii) maturation of Kasumi-1 cells. Kasumi-1 is an AML1-ETO positive human cell line that is often used in cell-based assays for hematopoietic maturation. Kasumi-1 cells are treated with the test compound and standard endpoints of hematopoietic maturation and apoptosis are analyzed as described (Wang et al., Cancer Res. 59:2766-2769, 1999; Moldenhauer et al., J. Leukoc. Biol. 76:623-633, 2004).

Compounds that have activity in at least one of these assays (cytology, in situ hybridization, or Kasumi-1 maturation), in addition to their activity in the original screen assay, can be considered of sufficient significance to warrant follow-up studies. Beyond their activities in the various biological assays, the apparent specificity, potency, and structural characteristics of the compounds can be considered in prioritizing initial hits for further study as follows:

i) apparent specificity. Embryos treated with each initial hit can be examined carefully by dissecting microscope for non-hematopoietic phenotypes including morphological changes, necrosis, developmental delay, and other signs of toxicity that can be observed by light microscopy. In addition, in vivo acridine orange staining can be performed to test for increased apoptosis in treated embryos (Pamg et al., Assay Drug Dev. Techno. 1:41-48, 2002). Small molecules that suppress the AML1-ETO phenotype without causing additional effects can be given priority over small molecules that cause pleiotropic effects.

ii) potency. Low potency is often associated with lack of specificity, while greater potency facilitates mechanism of action studies and increases therapeutic potential. Dose response curves can be determined for all initial hits as described (Peterson et al., Nat. Biotechno. 22:595-599, 2004), and priority can be given to compounds with lower EC50s. Ideally, compounds have EC50s of 100 nM or lower, and it may be possible to improve potency further using structure activity relationship (SAR) analysis as described (Perkins et al., Environ. Toxicol. Chem. 22:1666-1679, 2003; Tong et al., Environ. Toxicol. Chem. 22:1680-1695, 2003).

iii) structural characteristics. The chemical structures of all initial hits can be analyzed to determine whether they are related to other molecules with known biological functions, whether other structurally related molecules are present in the library or commercially available, and how amenable the structures are to synthesis and synthetic modification. SAR studies are easiest for structures for which numerous related molecules are commercially available and for structures that are easily synthesized. These structures will receive higher priority. In prioritizing compounds for further study, the greatest weight can be given to compounds that appear to be specific as defined above, because lack of specificity may confound follow-up studies. If multiple compounds appear to have adequate specificity, potency can next be considered, with the most potent molecule(s) being selected for further study. If multiple compounds have comparable specificity and potency, structural characteristics can be considered, giving priority to compounds that represent novel chemical classes and are amenable to synthetic manipulation.

Compounds identified using the screening methods described above can be used to treat patients that have or are at risk of developing AML. Treatment may be required only for a short period of time or may, in some form, be required throughout a patient's lifetime. Any appropriate route of administration can be employed to administer a compound identified as described above. For example, administration can be parenteral, intravenous, intra-arterial, subcutaneous, intramuscular, intraventricular, intracapsular, intraspinal, intracistemal, intraperitoneal, intranasal, by aerosol, by suppository, or oral. A therapeutic compound of the invention can be administered within a pharmaceutically-acceptable diluent, carrier, or excipient, in unit dosage form. Administration can begin before or after the patient is symptomatic. Methods that are well known in the art for making formulations are found, for example, in Remington's Pharmaceutical Sciences (18^{th} edition), ed. A. Gennaro, 1990, Mack Publishing Company, Easton, Pa. Further, determination of an appropriate dosage amount and regimen can readily be determined by those of skill in the art.

As discussed further below, we have shown that TIS11b plays a protective role in AML and, thus, the invention also includes methods of treating AML by increasing TIS11b levels. This can be accomplished by, for example, administration of agents (e.g., small organic molecules) that are identified in screening assays (e.g., in vitro or cell-based screening assays) as increasing expression and/or stability of TIS11b. In addition, TIS11b itself (or a nucleic acid molecule encoding TIS11b) can be used as a therapeutic agent. This can be achieved by, for example, administration of the protein or a gene therapy vector (e.g., a viral or plasmid vector) encoding the protein. In other approaches, ex vivo gene therapy is used. For example, cells (e.g., cells removed from a patient to be treated) are treated ex vivo to express TIS11b. In one example expression is induced by introduction (by, e.g., homologous recombination) of regulatory sequences that activate TIS11b expression. In another example, the TIS11b gene and appropriate regulatory sequences (e.g., inducible promoter elements) are introduced into the cells (e.g., by homologous recombination, stable transfection, and/or viral transduction). The cells are then administered to or implanted into a patient for treatment of AML, optionally, in combination with other approaches to treatment.

### EXPERIMENTAL RESULTS

Materials and Methods

Zebrafish Care and Embryo Collection

Zebrafish embryos were collected in Petri dishes and kept in a 23-28.5° C. incubator until reaching the desired stages. The stages (hours post-fertilization (hpf)) described in this report are based on the developmental stages of normal zebrafish embryos at 28.5° C.

Generation of Tg(hsp:AML1-ETO) Zebrafish Line

To construct pHSP/AML1-ETO, we first amplified a 4-kb zebrafish hsp70 promoter fragment from pHSP70-4 (Xiao et al., J. Neurosci. 23:4190-4198, 2003) and cloned it into the HindIII and PstI sites of the pG1 vector. Subsequently, the GFP fragment in pG1 was removed and replaced with the XbaI fragment containing the human AML1-ETO gene from pCS2cmv-RUNX1-CBF2T1 (Kalev-Zylinska et al., Development 129:2015-2030, 2002). The transgenic zebrafish were obtained by injecting linearized pHSP/AML1-ETO DNA into 1-cell stage zebrafish embryos. The zebrafish carrying the transgene were identified by fin-clipping and genotyping using PCR primers AML1-f, 5′-GGAAGAGGGAAAAGCTTCAC (SEQ ID NO:1), and ETO-r, 5′-GAGTAGTTGGGGGAGGTGG (SEQ ID NO:2).

Heat Treatment and Phenotyping

The Petri dishes containing zebrafish embryos were transferred from the growth temperature of 23-28.5° C. to a 37-42° C. incubator and incubated for 1 hour before returning them back to the normal temperature. The heat treatment may be repeated three to four times over 12-hour intervals as specified below. The percentages of embryos with phenotype were scored by visual inspection of the loss of circulating blood in the embryos after 40 hpf.

Morpholino Oligonucleotides and Microinjection

The morpholino antisense oligonucleotides hAML1-MO (5′-CTGGCATCTACGGGGATACGCATCA; SEQ ID NO:3), which targets the translation start codon of human AML1, and zTIS11b-MO (5′-ACTTTTCTCCATACCTTGTTGTTGA; SEQ ID NO:4), which targets the splice donor site of zebrafish TIS11b transcripts, were obtained from Gene-Tools, LLC. For microinjection, 500 μM hAML1-MO or 200 μM zTIS11b-MO in 0.3× Danieau's buffer (17 mM NaCl, 2 mM KCl, 0.12 mM MgSO₄, 1.8 mM Ca(NO₃)₂ and 1.5 mM HEPES, pH 7.6) were prepared and injected as described (Nasevicius et al., Nat. Genet. 26:216-220, 2000).

Fluorescence Microangiography

Fluorescence microangiography was done as described (Weinstein et al., Nat. Med. 1:1143-1147,1995).

Blood Extraction

Blood cells were collected from anesthetized wild-type and AML1-ETO embryos at 40 hpf by transferring live fish to phosphate buffered saline containing 50 U/ml heparin, 1% bovine serum albumin, and 0.006% tricaine. Tails were excised posterior to the yolk extension (at approximately the site of the posterior ICM) using a scalpel. Blood cells were extruded from the site of excision using the blunt edge of the scalpel and collected using a micropipette.

Cytology and Cytochemistry

For cytological analyses, blood cells collected from the zebrafish embryos were transferred onto glass slides by cytospin and stained by Protocol® Wright-Giemsa stain (Fisher Diagnostics) following manufacturer's instruction. To label red blood cells in the zebrafish embryos, whole-mount cytochemistry staining with diamino benzamidine was performed as previously described (Weinstein et al., Development 123:303-309, 1996).

RT-PCR Analysis

RNA was isolated with RNAqueous®-Micro (Ambion) from the blood samples of 10-20 zebrafish embryos. RNA was treated with DNaseI and then was subjected to cDNA synthesis using SuperScript™III (Invitrogen). One twentieth of the cDNA was used for real-time PCR by the SYBR green method (Applied Biosystems). mRNA levels were normalized to glyceraldehyde 3-phosphate dehydrogenase (GAPDH) expression. The fold of expression is depicted by the transcript levels in the heat-treated transgenic embryos relative to the transcript levels in the heat-treated wild-type embryos. Primer sequences used are as follows:

| | |
|---|---|
| cMYB-f, | |
| 5′-GTCATCGCCAGCTTTCTACC; | (SEQ ID NO:5) |
| cMYB-r, | |
| 5′-CTTTGCGATTACTGACCAACG; | (SEQ ID NO:6) |
| GATA1-f, | |
| 5′-GTCGTCCTATAGACACAGTC; | (SEQ ID NO:7) |
| GATA1-r, | |
| 5′-TTCTGGTAGATGGACGTGGAG; | (SEQ ID NO:8) |
| LMO2-f, | |
| 5′-CTTTCTGAAGGCCATCGAGC; | (SEQ ID NO:9) |
| LMO2-r, | |
| 5′-CAGAGTCCGTCCTGACCAAAC; | (SEQ ID NO:10) |
| SCL-f, | |
| 5′-GGAACAGTATGGGATGTATCC; | (SEQ ID NO:11) |
| SCL-r, | |
| 5′-GCAGGATCTCGTTCTTGCTG; | (SEQ ID NO:12) |
| PU.1-f, | |
| 5′;GAGATCTATCGACCACCAATG; | (SEQ ID NO:13) |
| PU.1-r, | |
| 5′-CTGGAAAGCGATGCACACTG; | (SEQ ID NO:14) |
| TIS11B-f, | |
| 5′-GCTAAGGCAGATCCATCCCTG; | (SEQ ID NO:15) |
| TIS11B-r, | |
| 5′-CACTTCTGTAGCAGGCGATCC; | (SEQ ID NO:16) |
| GAPDH-f, | |
| 5′-AGGCTTCTCACAAACGAGGA; | (SEQ ID NO:17) |
| GAPDH-r, | |
| 5′-GATGGCCACAATCTCCACTT. | (SEQ ID NO:18) |

Automated Imaging System

Plates containing wild-type and transgenic embryos were placed on the Universal Imaging Discovery-1 stage (Molecular Devices Corporation). We used MetaMorph software (Molecular Devices Corporation) to capture two successive stacks of 20 images for each embryo under transmitted light, and then performed digital subtraction of each frame of stack #1 from the corresponding frame of stack #2. This generated 20 “difference” images. We then added the 20 difference images to generate one “summed differences” image. The summed differences image from the transgenic embryo was blank, indicating that no detectable movement occurred during image capture. In contrast, the summed differences image from the wild-type embryo showed a bright signal that followed the path of circulation, indicating that these embryos possessed circulating hematopoietic cells.

Results

Induced Expression of AML1-ETO Causes an Accumulation of Hematopoietic Cells in the Transgenic Zebrafish Embryos

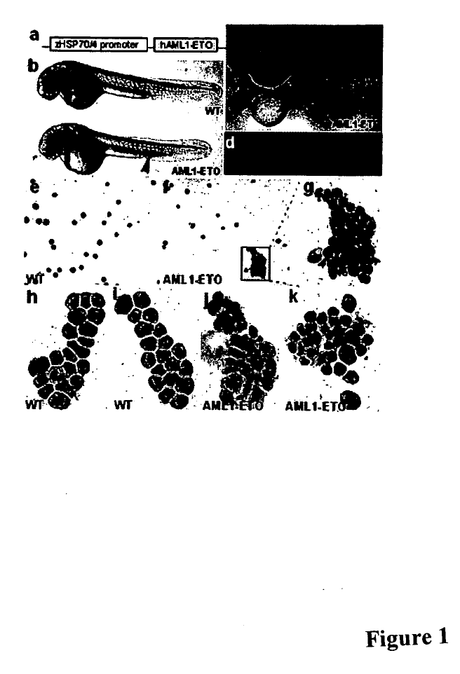

We sought to create a zebrafish model for studying AML1-ETO-mediated leukemogenesis by generating an inducible transgenic zebrafish line Tg(hsp:AML1-ETO) in which the human AML1-ETO transgene is under the control of the zebrafish hsp70 promoter (FIG. 1A). It has been shown that transgenes under the control of the zebrafish hsp70 promoter can be induced efficiently by incubating the transgenic fish at 37-42° C., instead of the normal water temperature of 28.5° C. (Xiao et al., J. Neurosci. 23:4190-4198, 2003). This inducible control allows the bypass of the potential embryonic lethality that has been observed in mouse models of AML1-ETO expression. As anticipated, we find that both hemizygous and homozygous Tg(hsp:AML1-ETO) adult fish are viable with no apparent phenotype, suggesting that without induction, the integration of the transgene does not affect normal zebrafish development.

To test the effect of AML1-ETO expression in zebrafish, we first crossed hemizygous Tg(hsp:AML1-ETO) fish with wild-type fish, and incubated the embryos at 37° C. for a total of four times at 4, 16, 24, and 36 hours post-fertilization (hpf) for one hour at each time. We then screened the embryos for any visible phenotypes at 44 hpf and genotyped each embryo individually. Due to their optical transparency, most of the internal components in the developing zebrafish embryos including the vascular system and blood cells can be observed simply under a dissecting microscope. Consistently, we found that heat-treated Tg(hsp:AML1-ETO) fish embryos have no circulating blood cells even though their hearts are beating. Moreover, the majority of the blood cells in these embryos accumulate in the intermediate cell mass (ICM) region, which lies along the trunk ventral to the dorsal aorta, as shown in live images and in embryos stained with diamino benzamidine (FIGS. 1B and 1C). On the other hand, wild-type embryos that have been subjected to the same heat shock treatment do not show any abnormality and establish robust circulation (FIGS. 1B and 1C).

In order to determine whether the accumulation of hematopoietic cells in the ICM is caused by a cardiovascular defect, we employed fluorescent microangiography to test cardiovascular structure and function. We found that fluorescein-coupled latex beads injected into the inflow tract of the atrium are able to perfuse the whole vascular system of the Tg(hsp:AML1-ETO) embryos and reveal a completely wild-type vascular pattern (FIG. 1D). This result indicates that functional hearts and patterned circulatory systems are present in the AML1-ETO-expressing embryos.

Interestingly, the ICM region is considered the ‘blood island’ in zebrafish embryos (Thompson et al., Dev. Biol. 197:248-269, 1998). During zebrafish development, the first wave of hematopoiesis, or primitive hematopoiesis, occurs within the ICM. Around 24 hpf, the differentiated hematopoietic cells then enter the circulatory system through the venous wall. Therefore, the accumulation of hematopoietic cells in the ICM is likely due to a hematopoietic defect that blocks development of mature cells competent to enter the circulation, rather than a defect in the circulatory system itself.

Immature Hematopoietic Blast Cells Accumulate in AML1-ETO-Expressing Zebrafish Embryos

The hallmark of AML is the arrest of myeloid differentiation with the expansion of immature hematopoietic progenitor cells. Using cytology, we determined that the hematopoietic cells that accumulate in the ICM of the AML1-ETO-expressing fish are dramatically enriched for immature blast-like cells reminiscent of human AML. Blood cells collected from anesthetized wild-type and Tg(hsp:AML1-ETO) embryos at 40 hpf after heat treatments were analyzed by Wright-Giemsa stain. As shown in FIGS. 1E-1F, blood from both wild-type and transgenic fish contains a mixture of individual cells and clusters of cells, although cell clusters are more prevalent in samples from the transgenic fish than in the samples from the wild-type fish. The blood cells from the wild-type fish are predominantly erythrocytes, with blast cells and other myeloid cells types only occasionally observed (FIGS. 1H and 1I). In contrast, blood from the transgenic fish is dramatically enriched for blast cells and other immature hematopoietic precursor cells (FIGS. 1G, 1J, and 1K). These data demonstrate that this inducible model faithfully reproduces the hallmark feature of human AML with accumulation and developmental arrest of hematopoietic blast cells.

The Zebrafish AML1-ETO Phenotype is Dependent on AML1-ETO Expression

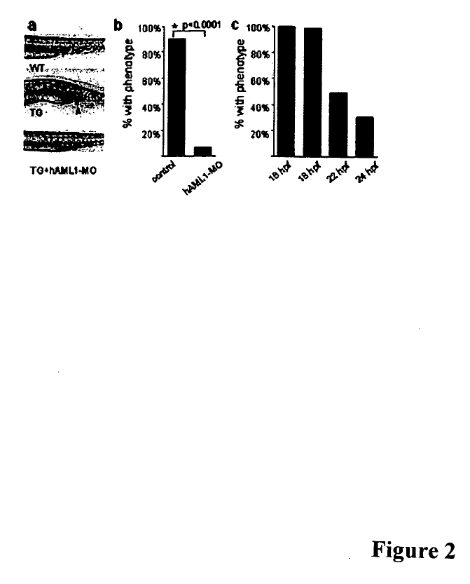

In AML1-ETO-expressing fish, the absence of circulating cells and the accumulation of non-circulating hematopoietic cells in the ICM are readily detected by eye. Therefore, circulation may be a simple surrogate phenotype for detecting the presence or absence of the AML1-ETO phenotype. To confirm that the loss-of-circulation phenotype is dependent on the inducible expression of AML1-ETO in the transgenic zebrafish, we tested whether this phenotype can be rescued by blocking AML1-ETO expression using an antisense morpholino oligonucleotide (hAML1-MO) complementary to the translation start site of the human AML1-ETO mRNA. Homogeneous transgenic embryo clutches were obtained from crosses between homozygous Tg(hsp:AML1-ETO) and wild-type fish. These embryos were heat treated at 4, 16, and 24 hpf, and were then scored at 44 hpf. As shown in FIG. 2A, the heat treatment regimen does not affect the circulation in the wild-type embryos. On the other hand, in the heat-treated Tg(hsp:AML1-ETO) fish embryos, most of the blood cells accumulate in the ICM, especially at the location close to the end of the yolk extension, and fail to enter circulation. However, injection of HAML1-MO into 1-cell stage embryos restores the circulation in the transgenic embryos. We found that while around 90% of the uninjected AML1-ETO-expressing fish embryos exhibit no circulating blood cells, less than 10% of the morpholino-injected transgenic embryos exhibit the phenotype (FIG. 2B). These data show that the phenotype observed in the Tg(hsp:AML1-ETO) fish is AML1-ETO dependent.

One of the advantages of this zebrafish model is the ability to control the timing and the extent of AML1-ETO expression. To investigate when the disruption of hematopoietic programming mediated by AML1-ETO occurs, we have induced AML1-ETO expression during various stages of embryonic development. We found that even though expression of AML1-ETO at 18 hpf results in almost 100% penetrance, expression of AML1-ETO at 22 hpf significantly reduces the percentage of embryos exhibiting the phenotype (FIG. 2C), suggesting that there is a limited window of time during embryonic development when AML1-ETO expression is able to cause a dramatic accumulation of blast cells in the ICM.

Retinoic Acid can Partially Rescue the AML1-ETO Phenotype

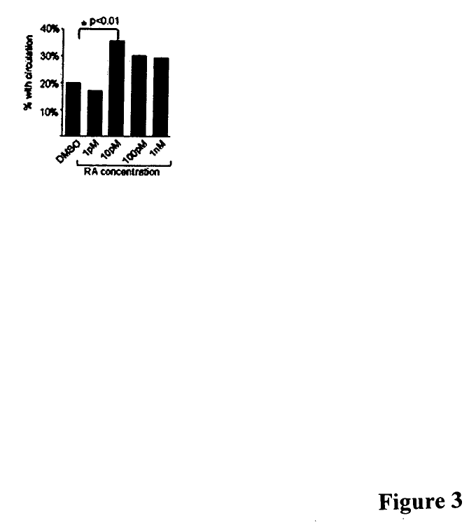

Zebrafish embryos readily absorb small molecules from the surrounding medium, rendering them a powerful tool to assess pharmacological efficacy (Peterson et al., Methods Cell. Biol. 76:569-591, 2004; Zon et al., Nat. Rev. Drug Discov. 4:35-44, 2005). It has been shown that the all-trans retinoic acid (ATRA) signaling pathway plays a role in myeloid differentiation, and ATRA is highly effective at treating acute promyelocytic leukemia (Dulaney et al., Ann. Pharmacother. 27:211-214, 1993). While ATRA is generally not very effective in differentiating t(8;21) leukemic cells, complete remission of a patient with t(8;21) translocation has been reported (Chen et al., Chin. Med. J. (Engl). 115:58-61, 2002). We tested the efficacy of ATRA in reversing the zebrafish AML1-ETO phenotype. In this experiment, we heat-shocked the embryos three times instead of four times at 37° C. in order to reduce the phenotypic penetrance in the Tg(hsp:AML1-ETO) fish embryos to around 80%. Meanwhile, ATRA was added at 24 hpf. We scored the percentage of embryos with circulating blood cells, and found that 10 pM of ATRA was able to increase the percentage of embryos possessing circulation from 20% to 36% (p=0.0085) (FIG. 3). The degree of rescue did not increase at higher concentrations, possibly due to competing toxicities that emerge at higher doses. Consistent with this idea, we found that at 100 nM concentration, the embryos exhibit extreme pericardial edema and a lack of circulation likely due to a previously recognized cardiac defect (Stainier et al., Dev. Biol. 153:91-101, 1992). These data demonstrate that the zebrafish AML1-ETO phenotype can be reversed using a pharmacological agent, and that this model can be used for identifying small molecule modifiers of AML.

Transcriptional Changes in the Blood of AML1-ETO Transgenic Zebrafish Embryos Parallel those Observed in Human AML

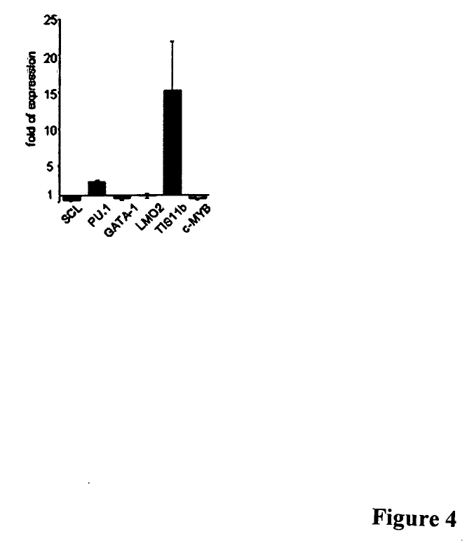

Significant conservation exists between zebrafish and human hematopoiesis at the molecular level (Davidson et al., Oncogene 23:7233-7246, 2004). To test whether the transcriptional changes in the zebrafish AML1-ETO model are consistent with those in human AML patients, we extracted blood samples from either wild-type or AML1-ETO-expressing fish embryos at 40 hpf, and used real-time PCR analysis to quantify the expression levels of several hematopoietic genes in these blood samples. The change in expression was obtained by comparing the amount of each transcript in the transgenic samples with the amount in the wild-type samples after each had been normalized to the level of glyceraldehyde-3-phosphate dehydrogenase (GAPDH) transcript in the same sample. As shown in FIG. 4, we found that the expression of c-MYB, a transcription factor required for differentiation of definitive hematopoietic cell types, and SCL, a marker for hematopoietic stem cells, are reduced to about 37% and 29% of normal expressions in the AML1-ETO-expressing samples. c-MYB is a marker of definitive hematopoietic cells in zebrafish (Thompson et al., Dev. Biol. 197:248-269, 1998), so its down regulation in this model suggests an inhibitory role for AML1-ETO on zebrafish definitive hematopoiesis. Interestingly, the expression of AML1-ETO in mouse embryos also results in a failure of definitive hematopoiesis (Okuda et al., Blood 91:3134-3143, 1998). In addition, SCL gene expression is not detectable in Kasumi-1 cells, a human myelocytic leukemia cell line that expresses AML1-ETO, nor in all four leukemia samples from patients harboring the t(8;21) translocation tested by Bennett et al. (Blood 98:643-651, 2001). Therefore, the reduction in c-MYB and SCL expression demonstrates similarity between the zebrafish AML1-ETO phenotype and human AML.

The expression level of PU.1, a master regulator of myeloid cells, is increased 2.7 fold in the zebrafish AML1-ETO model. PU.1 plays a critical role in myeloid development and is a marker for myeloid cells (Lieschke et al., Dev. Biol. 246:274-295, 2002; Lieschke et al., Dev. Biol. 246:274-295, 2002). Thus, our data suggest an increase of myelopoiesis in the AML1-ETO-expressing embryos as in human AML. GATA-1, a transcription factor expressed in erythrocytes, and LMO2, a transcription factor implicated in early commitment to the hematopoietic lineage, are only mildly affected. Of the genes tested, the most dramatic change is a 15-fold increase in TIS11b expression. Increased expression of TIS11b has also been shown by ectopically expressing AML1-ETO in a myeloid precursor cell line (L-G cells) and in human leukemia samples with the t(8;21) translocation (Shimada et al., Blood 96:655-663, 2000). These results show that in addition to the cytological similarities between our model and human AML, we can detect changes in gene expression that parallel those found in human AML.

TIS11b Knockdown in AML1-ETO-expressing Embryos Enhances the AML1-ETO Phenotype

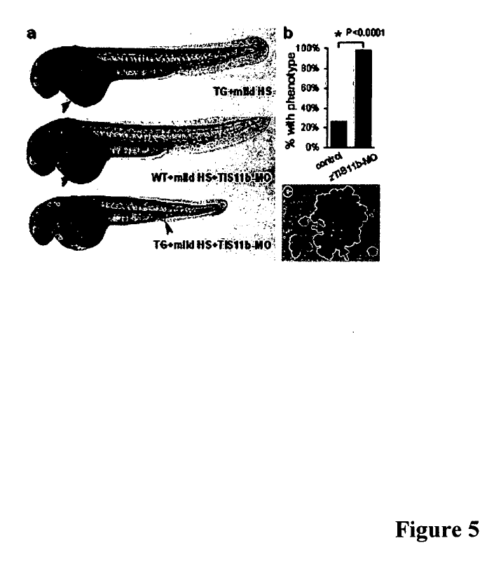

The upregulation of TIS11b had been hypothesized to contribute to AML pathogenesis, but this hypothesis had not been tested. To elucidate the role of TIS11b in the AML1-ETO phenotype, we knocked down TIS11b by injecting an antisense morpholino oligonucleotide complementary to the splice acceptor site of the zebrafish TIS11b gene (zTIS11b-MO). We found that, instead of rescuing the phenotype, TIS11b knockdown strongly potentiates the ability of AML1-ETO to cause the phenotype. As shown in FIGS. 5A and 5B, under a mild heat treatment, while less than 30% of uninjected Tg(hsp:AML1-ETO) embryos exhibit the lack of circulation phenotype, 98% of the zTIS11b-MO-injected Tg(hsp:AML1-ETO) fish embryos exhibit the AML1-ETO phenotype. This is not caused by knocking down the normal level of TIS11b expression because all wild-type embryos injected with zTIS11b-MO still have circulation after the same heat treatment (FIG. 5A). In addition to the loss of circulation, we have also detected an accumulation of hematopoietic blast cells in the Tg(hsp:AML1-ETO) embryos injected with zTIS11b-MO by cytological analysis (FIG. 5C). These data show that the increased expression of TIS11b partially compensates for the pathogenic effect of AML1-ETO expression. This is the first demonstration of a protective role for TIS11b in AML, and highlights the rapidity with which a candidate drug target or disease modifier can be evaluated in this model' system.

The Zebrafish AML1-ETO Phenotype can be Detected Automatically

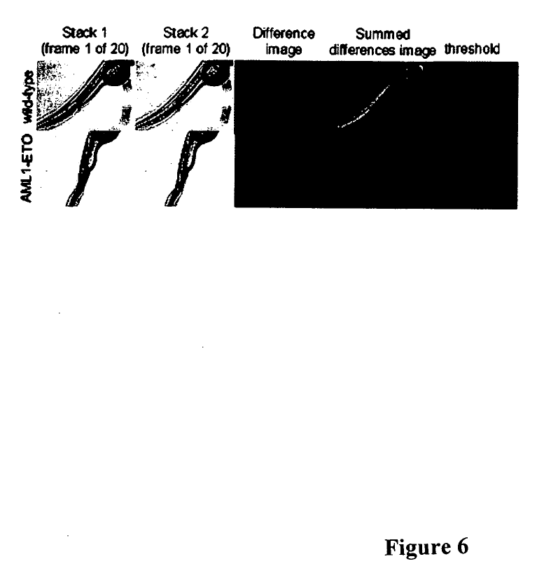

To expand the utility of our model and to adapt this model into a high-throughput platform, we have shown that the zebrafish AML1-ETO phenotype can be scored digitally using an automated screening system. We exploited a digital subtraction methodology based on the presence of moving blood cells in the wild-type but not the AML1-ETO-expressing fish. Plates containing wild-type and transgenic embryos were placed on the Universal Imaging Discovery-1 stage, and two successive stacks of 20 images were captured for each embryo using transmitted light (FIG. 6, columns 1-2). Using MetaMorph software, we then performed digital subtraction of each frame of stack #1 from the corresponding frame of stack #2. This generated 20 “difference” images. These difference images were blank except for pixels that differed in intensity between the subtracted frames (FIG. 6, column 3). We then added the 20 difference images to generate one “summed differences” image (FIG. 2, column 4). The summed differences images from the transgenic embryos were blank, indicating that no detectable movement occurred during image capture. In contrast, the summed differences images from the wild-type embryos showed bright signals that followed the path of circulation, indicating that these embryos possessed circulating hematopoietic cells. The MetaMorph object recognition software was then used to identify and determine size parameters of the signal. Using a preset threshold and size parameter, the path of circulation can be determined and distinguished from the noise (FIG. 2, column 5). These data demonstrate that zebrafish embryos exhibiting the AML1-ETO phenotype can readily be distinguished from embryos with a wild-type phenotype using digital subtraction and object recognition. This optical assay for zebrafish circulation can be fully automated and used to systematically detect the presence of circulation in zebrafish distributed into wells of 96-well plates.

All references cited above are incorporated by reference herein in their entirety. Other embodiments are within the scope of the following claims.

What is claimed is:

1. a method for identifying an agent that can be used in the treatment of acute: myelogenous leukemia (AML), the method comprising:
(i) providing a zebrafish that expresses a gene product that induces a phenotype characteristic of AML,
(ii) contacting the zebrafish with a candidate agent, and
(iii) analyzing the effects of the agent on an AML-related phenotype of the zebrafish, wherein detection of an improvement in the phenotype indicates identification of an agent that can be used in the treatment of AML.

2. The method of claim 1, wherein the gene product blocks myeloid differentiation in AML.

3. The method of claim 1, wherein expression of the gene product is under the control of an inducible promoter, and expression of the gene product is induced prior to contacting the zebrafish with the candidate agent

4. The method of claim 1, wherein the gene product is a protein.

5. The method of claim 4, wherein the protein is a fusion protein comprising sequences of AML1 and eight twenty one (ETO).

6. The method of claim 5, wherein the fusion protein comprises the DNA binding domain of AML 1.

7. The method of claim 5, wherein the sequences of AML1 and ETO are human sequences.

8. The method of claim 1, wherein the AML-related phenotype is loss of circulation.

9. The method of claim 1, wherein the AML-related phenotype is accumulation of hematopoietic cells in the intermediate cell mass (ICM).

10. The method of claim 1, wherein the AML-related phenotype is loss of hematopoietic cell maturation as detected by analysis of a hematopoietic marker.

11. The method of claim 10, wherein the hematopoietic marker is PU.1, GATA-1, myeloid-specific peroxidase (MPO), or SCL.

12. The method of claim 1, wherein the zebrafish is an embryo.

13. The method of claim 3, wherein expression of the gene product is induced at 4-24 hours post fertilization.

14. The method of claim 1, wherein the AML-related phenotype is monitored at 24-72 hours post fertilization.

15. The method of claim 3, wherein the inducible promoter is a heat shock protein promoter, and induction of expression is achieved by incubation of the zebrafish at an elevated temperature.

16. The method of claim 1, wherein the agent is a small organic molecule.

17. The method of claim 1, further comprising the analysis of multiple zebrafish, which are present in separate wells of a multi-well plate, and are contacted with different candidate agents.

18. The method of claim 17, comprising the use of an automated system to screen the phenotypes of the zebrafish.

19. A zebrafish comprising a gene encoding a gene product that induces a phenotype characteristic of AML.

20. The zebrafish of claim 19, wherein the gene product is an AML1-ETO fusion protein.

21. The zebrafish of claim 19, wherein expression of the gene product is under the control of an inducible promoter.

22. The zebrafish of claim 20, wherein the AML1-ETO fusion protein comprises the DNA binding domain of AML1.

23. The zebrafish of claim 20, wherein the AML1-ETO fusion protein comprises human sequences.

24. The zebrafish of claim 21, wherein the inducible promoter is a heat shock protein promoter.

25. The zebrafish of claim 19, wherein the zebrafish is mature.

26. The zebrafish of claim 19, wherein the zebrafish is an embryo.

27. A method of identifying a therapeutic agent, the method comprising:
(i) providing a zebrafish exhibiting a phenotype characteristic of a disease or condition,
(ii) incubating the zebrafish in the presence of a candidate therapeutic agent, and
(iii) monitoring the phenotype of the zebrafish using an automated system, wherein detection of an improvement in the phenotype indicates the identification of a therapeutic agent that can be used in the treatment of the disease or condition.

28. The method of claim 27, wherein the phenotype characteristic of the disease or condition is due to a mutation in the zebrafish.

29. The method of claim 27, wherein the phenotype characteristic of the disease or condition is due to induction of expression of a transgene encoding a protein that causes the phenotype characteristic of the disease or condition.

30. A method of treating AML in a patient, the method comprising increasing TIS11b levels in the patient.

31. The method of claim 30, wherein TIS11b is administered to the patient.

32. The method of claim 30, wherein a nucleic acid molecule encoding TIS11b is administered to the patient.

33. A method for identifying an agent that can be used in the treatment of AML, the method comprising introducing a candidate agent into an expression system comprising a gene encoding TIS11b, and determining whether the candidate agent increases expression, stability, and/or activity of TIS11b.

34. The method of claim 33, wherein the expression system is in a cell.
